(19) United States
(12) Patent Application Publication
(10) Pub. No.: US 2006/0013902 A1
(43) Pub. Date: Jan. 19, 2006

(54) Pharmaceutical products for treating neoplastic disease and inflammation

(75) Inventor: Najla Guthrie, London (CA)

Correspondence Address: DAVIDSON, DAVIDSON & KAPPEL, LLC, 485 SEVENTH AVENUE, 14TH FLOOR, NEW YORK, NY, 10018, US

(73) Assignee: KGK Synergize Inc., London (CA)

(21) Appl. No.: 11/135,694

(22) Filed: May 24, 2005

Related U.S. Application Data
(60) Provisional application No. 60/574,487, filed on May 26, 2004.

(51) Int. Cl. A61K 31/7048; A61K 31/353; A61K 36/752; A61K 31/355

(52) U.S. Cl. 424/736; 514/27; 514/456; 514/458

(57) ABSTRACT

Disclosed in certain embodiments is a pharmaceutical formulation and a functional food comprising a pharmaceutical ingredient comprising an active agent combination comprising flavonoids and tocotrienols in a ratio of about 75:25 to about 95:5 and at least one pharmaceutically acceptable excipient and methods to treat neoplastic diseases and inflammation.

### RELATED APPLICATIONS

This application claims priority to U.S. Provisional Application No. 60/574,487, filed May 26, 2004, which is hereby incorporated by reference.

### FIELD OF THE INVENTION

The present invention is directed to compositions comprising flavonoids and tocotrienols and method of treating cancer and inflammation.

### BACKGROUND OF THE INVENTION

Cancer is the second leading cause of death in the United States, after heart disease (Boring, C. C. et al., 1993, CA Cancer J. Clin. 43:7), and develops in one in three Americans, and one of every four Americans dies of cancer. Cancer can be viewed as a breakdown in the communication between tumor cells and their environment, including their normal neighboring cells. Signals, both growth-stimulatory and growth-inhibitory, are routinely exchanged between cells within a tissue. Normally, cells do not divide in the absence of stimulatory signals, and likewise, will cease dividing in the presence of inhibitory signals. In a cancerous, or neoplastic state, a cell acquires the ability to “override” these signals and to proliferate under conditions in which normal cells would not grow.

In addition to unhindered cell proliferation, cells must acquire several traits for tumor growth to occur. For example, early on in tumor development, cells must evade the host immune system. Further, as tumor mass increases, the tumor must acquire vasculature to supply nourishment and remove metabolic waste. Additionally, cells must acquire an ability to invade adjacent tissue, and ultimately cells often acquire the capacity to metastasize to distant sites.

Cancer of the breast is the most common form of malignant disease occurring among women of the Western World, and it is the most common cause of death among those who are between 40 and 45 years of age.

In North American women, characteristics that are associated with a threefold to fourfold increase in risk for breast cancer include (1) first-degree female family members (mothers and sisters) who had breast cancer, (2) prior breast cancer, (3) nulliparity, (4) age greater than 30 years at first pregnancy and (5) early menarche or late menopause (Sattin, R. W. et al., 1985, JAMA 253:1908). International studies have demonstrated a positive correlation between per capita consumption of fat and alcohol (Schatzkin A. et al., 1987, N. Engl. J. Med. 316:1169) and the incidence of breast cancer. (Carroll, K. K., 1980, J. Env. Pathol. Tox. 3:253-271). Several studies have linked the consumption of fresh fruits and vegetables, and vitamin E with reduced risk of developing cancer, including breast cancer (Steinmetz, K. A. et al., 1991, Cancer Causes Control 2:427-442). Although this protective effect has been generally attributed to the antioxidant capacities of vitamin C and beta-carotene present in these foods, it may be related to other phytochemical constituents such as citrus limonoids and flavonoids. The use of limonoids, flavonoids or tocotrienols alone or in combination with each other or with a cancer chemo-therapeutic agent has not been reported for the prevention and treatment of neoplastic diseases.

The present invention provides a number of different citrus limonoids comprising, but not limited to, limonin, nomilin, limonin glucoside or glucoside mixture, flavonoids comprising nobiletin or tangeretin and tocotrienol comprising alpha-tocotrienol, gamma-tocotrienol or delta-tocotrienol.

Cancers that can be prevented and/or treated by the compositions and methods of the present invention include, but are not limited to, human sarcomas and carcinomas, e.g. carcinomas, e.g., colon carcinoma, pancreatic cancer, breast cancer, ovarian cancer, prostate cancer, fibrosarcoma, myxosarcoma, liposarcoma, chondrosarcoma, osteogenic sarcoma, chondroma, angiosareoma, endotheliosarcoma, lymphangiosareoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. Specific examples of such cancers are described in the sections below.

Inflammation is typically associated with: (1) redness, (2) swelling, (3) heat and (4) pain, with a possible fifth sign being loss of function of the affected part. While injury triggers a complex series of events, many of which occur simultaneously and are interrelated in a variety of ways, it is known that small blood vessels participate in an important way in the induction of inflammation. In fact, inflammation is one of the body's valuable defense mechanisms and is generally thought of as having three phases: the degenerative phase, the vascular phase, and the healing phase. See Klein, “Defense Reactions in Action”, Immunology, The Science of Self-Nonself Discrimination, Chapter 14, 577-84 (1982), the disclosure of which is hereby incorporated by reference

The present invention relates to compositions and methods for the prevention and treatment of neoplastic diseases and/or inflammation with combinations of flavonoids and tocotrienols. Flavonoids are polyphenolic compounds that occur unbiquitiously in plant foods especially in orange, grapefruit, and tangerine. Tocotrienols are present in palm oil and are a form of vitamin E having an unsaturated side chain.

### SUMMARY OF THE INVENTION

It is an object of the present invention to provide a pharmaceutical ingredient, formulation or functional food for treating and/or preventing neoplastic disease and/or inflammation comprising flavonoids and tocotrienols.

It is a further object of the present invention to provide methods of treating neoplastic disease and/or inflammation by administering a pharmaceutical ingredient, formulation or functional food comprising flavonoids and tocotrienols.

It is another object of the invention is to a a pharmaceutical ingredient, formulation or functional food and methods to treat neoplastic disease and/or inflammation by utilizing flavonoids and tocotrienols, wherein the a pharmaceutical ingredient, formulation or functional food have low levels of synephrine.

Certain of the above objects of the invention can be achieved by the present invention which in certain embodiments is directed to a pharmaceutical ingredient comprising an active agent combination comprising polymethoxylated flavonoids and tocotrienols in a ratio of about 75:25 to about 95:5, the pharmaceutical ingredient selected from the group consisting of an essence oil isolated from a citrus fruit, a peel oil isolated from a citrus fruit, a peel isolated from a citrus fruit, decharacterized citrus fruit, and combinations thereof.

In certain embodiments, the invention is directed to a pharmaceutical formulation or functional food comprising a pharmaceutical ingredient comprising an active agent combination comprising flavonoids and tocotrienols in a ratio of about 75:25 to about 95:5 and at least one pharmaceutically acceptable excipient.

In certain embodiments, the invention is directed to a methods of treating neoplastic disease and/or inflammation by administering a pharmaceutical ingredient, formulation or functional food disclosed herein.

The term “essence oil” refers to the oil-soluble components (e.g., fraction) remaining after evaporation of a fruit juice.

The term “peel oil” refers to oil isolated from the peel of a citrus fruit.

The term “peel” refers to the peel of a citrus fruit which, for purposes of the present invention, may be e.g., dried, shredded, or pelletized.

The term “citrus fruit” refers to a fruit from the genus Citrus that includes, e.g., orange, lemon, lime, tangerine, grapefruit (e.g., pink grapefruit, red peel grapefruit) and, in particular, citrus aurentium.

The term “decharacterized fruit” refers to fruit from which the juice has been extracted. The decharacterized fruit can be in the form of, for example, a mash or presscake. The term “Tomah presscake” refers to a particularly preferred presscake described in U.S. Pat. Nos. 5,320,861 and 5,320,861 which contains higher levels of desirable phytochemicals than are present in presscake made via conventional methods. In particular, decharacterized cranberry fruit in the form of “Tomah presscake” contains higher levels of anthocyanins, phenolic acids and proanthocyanidins than that found in presscake produced through conventional methods. For example, the anthocyanin content is typically 30% or greater of that present in native cranberry fruit, the phenolic acid content is typically 8% or greater of that present in native cranberry fruit and the proanthocyanidin content is typically 60% or greater of that present in native cranberry fruit.

The term “isolated” refers to the removal or change of a composition or compound from its natural context.

The term “flavonoid” includes, but is not limited to polymethoxylated flavonoids and refers to any member of the group of aromatic, oxygen-containing, heterocyclic pigments found in the derivatives of the invention and includes for example members of the chemical subgroups 1) catechins, 2) leucoanthocyanidins and flavanones, 3) flavanins, flavones, and anthocyanins, and 4) flavonols. In preferred embodiments, a flavonoid includes, e.g., a proanthocyanidin, flavan-3-ol, anthocyanin, or flavanol. The flavonoid can include e.g., naringenin, hesperetin, nobiletin, and/or tangeretin.

The term “tocotrienol” refers to any tocopherol (T) or tocotrienol (T3) compound, for example, .alpha.-tocopherol, .gamma.-tocopherol, .delta.-tocopherol, .alpha.-tocotrienol, .gamma.-tocotrienol, .delta.-tocotrienol, or a combination thereof, that is present in measurable levels in the fruit derivatives of the invention.

The term “pharmaceutical ingredient” means a therapeutic composition which can be optionally combined with pharmaceutically acceptable excipients to provide a pharmaceutical formulation or dosage form.

The term “pharmaceutical formulation” means a pharmaceutical ingredient in combination with at least one pharmaceutically acceptable excipient. The formulation can be administered by any acceptable route, e.g., oral in any acceptable form, e.g., a tablet or capsule.

The term “functional food” for purposes of the present invention are any edible or drinkable foods or dietary components (e.g., juices, bakery products, applesauce, etc) that are fortified or enhanced with flavonoids and tocotrienols as disclosed herein. The functional food can be, e.g., solid, liquid, semisolid, or a combination thereof. The term “functional food” also encompasses edible and drinkable nutritional supplements.

### DETAILED DESCRIPTION OF THE INVENTION

In certain embodiments, the present invention is directed to a pharmaceutical ingredient comprising an active agent combination comprising polymethoxylated flavonoids and tocotrienols in a ratio of about 75:25 to about 95:5, the pharmaceutical ingredient selected from the group consisting of an essence oil isolated from a citrus fruit, a peel oil isolated from a citrus fruit, a peel isolated from a citrus fruit, decharacterized citrus fruit, and combinations thereof.

In certain embodiments, the active agent combination comprises flavonoids and tocotrienols in a ratio of about 90:10; in a ratio of about 80:20; or in a ratio of about 95:5.

In certain embodiments, the pharmaceutical ingredient of the present invention comprising from about 50% to about 90% of flavonoids and tocotrienols; from about 60% to about 80% of the active agent combination; or about 70% of the active agent combination.

In certain embodiments, the pharmaceutical ingredient contains less than about 1% synephrine; less than about 0.5% synephrine; or less than 0.1% synephrine.

The flavonoid of the present invention can be a polymethoxylated flavonoid. In certain embodiments, the flavonoid comprises a member selected from the group considting of naringenin, hesperetin, nobiletin, tangeretin and combinations thereof.

The tocotrienol of the present invention can be, e.g., selected from the group consisting of alpha-tocotrienol, gamma-tocotrienol, delta-tocotrienol, and combinations thereof.

In certain embodiments, the invention is directed to a pharmaceutical formulation or functional food comprising a pharmaceutical ingredient comprising an active agent combination comprising flavonoids and tocotrienols in a ratio of about 75:25 to about 95:5 and at least one pharmaceutically acceptable excipient.

In certain embodiments, the pharmaceutical formulation or functional food of the present invention comprises a pharmaceutical ingredient selected from the group consisting of an essence oil isolated from a citrus fruit, a peel oil isolated from a citrus fruit, a peel isolated from a citrus fruit, decharacterized citrus fruit, and combinations thereof.

In certain embodiments, the pharmaceutical ingredient of the formulation of the present invention comprises is in an effective amount to treat a human subject at risk of or suffering from a neoplastic disease and/or inflammation.

In certain embodiments, the pharmaceutical formulation of the present invention is suitable for administration intravenously, intraperitoneally, subcutaneously, intramuscularly, intrathecally, orally, rectally, topically, or by inhalation.

In certain embodiments, the pharmaceutical formulation of the present invention is in the form of a tablet, a capsule, a solution, a liquid, a suspension, or an emulsion.

In certain embodiments wherein the invention is in the form of a functional food, the functional food is in the form of edible or drinkable compositions, e.g., foodstuffs such as chewable or edible bars, confectionary products (e.g., chocolate bars), cookies, juice drinks, baked or simulated baked goods (e.g., brownies), biscuits, lozenges or chewing gum. Preferred chewable or edible bars include chocolate bars and brownies. Such foods are beneficial as they provide the benefits of flavonoids and tocotrienols as disclosed above and also provide the benefit of relieving hunger or fatigue. Such functional foods can be particularly useful to people participating in sports or other forms of exercise.

The functional foods may also be in the form of, for example, butter, margarine, bread, cake, milk shakes, ice cream, yogurt and other fermented milk product.

The functional food can also be in the form of a powder to be sprinkled on meats, salads or other foods. They may be incorporated into solid foods such as candy bars, cereals, health bars and other comestibles.

Other forms if the functional foods can be breakfast cereals such as grain flakes or muesli.

In certain embodiments, the pharmaceutical formulation or functional food of the present invention comprises from about 60 mg of the tocotrienol and about 560 mg of the flavonoid per unit dose; from about 10 mg to about 80 mg of the tocotrienol and from about 150 mg to about 750 mg of the flavonoid per unit dose; or about 30 mg of the tocotrienol and about 270 mg of the flavonoid per unit dose.

In the methods of the present invention, the daily dose of the active agents can be, e.g., from about 60 mg of the tocotrienol and about 560 mg of the flavonoid; from about 10 mg to about 80 mg of the tocotrienol and from about 150 mg to about 750 mg of the flavonoid; or about 30 mg of the tocotrienol and about 270 mg of the flavonoid.

In the methods of the present invention, the flavonoids and the tocotrienols can be administered in the same dosage form or functional food or in separate dosage forms or functional foods. Further, the flavonoids and tocotrienols can be administered by the same route of administration or by different routes of administration.

The pharmaceutical formulations of the present invention can be prepared as oral, sublingual, inhaled, subcutaneous, intramuscular, intravenous, transdermal, and formulations for local or rectal administration. Oral formulations can be in the form of, e.g., tablets, gel capsules, powders, granules and oral solutions or suspensions, sublingual and buccal administration forms.

When a solid composition is prepared in the form of tablets or gel capsules, a mixture of pharmaceutical excipients which can be composed of diluents such as, for example, lactose, microcrystalline cellulose, starch, dicalcium phosphate, binders such as, for example, polyvinylpyrrolidone, hydroxypropylmethylcellulose, crumbling agents such as crosslinked polyvinylpyrrolidone, crosslinked carboxymethyl-cellulose, flow agents such as silica or talc, and lubricants such as magnesium stearate, stearic acid, glyceryl tribehenate or sodium stearyl fumarate, is added to the micronized or non-micronized active principle.

Wetting agents or surfactants such as sodium lauryl sulphate, polysorbate 80 or poloxamer 188 can be added to the formulation.

The tablets can be prepared by various techniques: direct tabletting, dry granulation, wet granulation, hot-melt.

The tablets may be uncoated coated (e.g., with sucrose) or coated with various polymers (e.g., hydroxypropylmethylcellulose) or other suitable materials.

The tablets can have immediate, delayed or sustained release by preparing matrices or by using coatings.

The gel capsules can be soft or hard, and coated with film or otherwise, so as to have immediate, sustained or delayed activity (for example via an enteric form).

Oral formulations can also be prepared as liquid or semi-solid formulations, as e.g., a preparation in the form of a syrup or elixir can contain the active principle together with a sweetener, preferably a calorie-free sweetener, methyl paraben and propyl paraben as antiseptic agent, as well as a flavouring agent and a suitable colorant.

Water-dispersible powders or granules can contain the active principle as a mixture with dispersants, wetting agents or suspending agents, such as polyvinylpyrrolidone, as well as with sweeteners or flavour enhancers.

For rectal administration, use is made of suppositories which are prepared with binders that melt at the rectal temperature, for example cocoa butter or polyethylene glycols.

Aqueous suspensions, isotonic saline solutions or sterile, injectable solutions which contain pharmacologically compatible dispersants and/or solubilizing agents, for example propylene glycol, are used for parenteral or intranasal administration.

Thus, in order to prepare an aqueous solution which can be injected intravenously, a co-solvent such as, for example, an alcohol such as ethanol or a glycol such as polyethylene glycol or propylene glycol, and a hydrophilic surfactant such as polysorbate 80 or poloxamer 188 can be used. To prepare an injectable oily solution for intramuscular administration, the active principle can be dissolved with a triglyceride or a glycerol ester.

Creams, ointments, gels, transdermal patches and sprays can be used for local administration. Patches in multilaminar or reservoir form in which the active principle can be in alcoholic solution, and sprays can be used for transdermal administration.

An aerosol containing, for example, sorbitan trioleate or oleic acid as well as trichlorofluoromethane, dichlorofluoromethane, dichlorotetrafluoroethane, freon substitutes or any other biologically compatible propellent gas is used for administration by inhalation; a system containing the active principle alone or combined with an excipient, in powder form, can also be used.

The active principle can also be formulated in the form of microcapsules or microspheres, optionally with one or more supports or additives.

Among the sustained-release forms which are useful in the case of chronic treatments, it is possible to use implants. These can be prepared in the form of an oily suspension or in the form of a suspension of microspheres in an isotonic medium.

1. A pharmaceutical formulation comprising a pharmaceutical ingredient comprising an active agent combination comprising flavonoids and tocotrienols in a ratio of about 75:25 to about 95:5 and at least one pharmaceutically acceptable excipient, wherein the pharmaceutical ingredient is in an effective amount to treat a human subject at risk of or suffering from a neoplastic disease.

2. A pharmaceutical formulation comprising a pharmaceutical ingredient comprising an active agent combination comprising flavonoids and tocotrienols in a ratio of about 75:25 to about 95:5 and at least one pharmaceutically acceptable excipient, wherein the pharmaceutical ingredient is in an effective amount to treat a human subject at risk of or suffering from inflammation.

3. The pharmaceutical formulation of claim 1, wherein the pharmaceutical ingredient is selected from the group consisting of an essence oil isolated from a citrus fruit, a peel oil isolated from a citrus fruit, a peel isolated from a citrus fruit, decharacterized citrus fruit, and combinations thereof.

4. The pharmaceutical formulation according to claim 1, wherein the formulation is suitable for administration intravenously, intraperitoneally, subcutaneously, intramuscularly, intrathecally, orally, rectally, topically, or by inhalation.

5. The pharmaceutical formulation according to claim 4, wherein the formulation is suitable for administration orally.

6. The pharmaceutical formulation according to claim 5, wherein the formulation is in the form of a tablet, a capsule, a gel capsule, a solution, a liquid, a suspension, or an emulsion.

7. The pharmaceutical formulation according to claim 1, comprising from about 10 mg to about 80 mg of the tocotrienol and from about 150 mg to about 750 mg of the flavonoid per unit dose.

8. The pharmaceutical formulation according to claim 1, comprising about 30 mg of the tocotrienol and about 270 mg of the flavonoid per unit dose.

9. The pharmaceutical formulation according to claim 1, comprising about 60 mg of the tocotrienol and about 560 mg of the flavonoid per unit dose.

10. A functional food comprising an edible solid or liquid; and a pharmaceutical ingredient comprising an active agent combination comprising flavonoids and tocotrienols in a ratio of about 75:25 to about 95:5 and at least one pharmaceutically acceptable excipient, wherein the pharmaceutical ingredient is in an effective amount to treat a human subject at risk of or suffering from a neoplastic disease.

11. A functional food comprising an edible solid or liquid; and a pharmaceutical ingredient comprising an active agent combination comprising flavonoids and tocotrienols in a ratio of about 75:25 to about 95:5 and at least one pharmaceutically acceptable excipient, wherein the pharmaceutical ingredient is in an effective amount to treat a human subject at risk of or suffering from inflammation.

12. The functional food of claim 10, wherein the pharmaceutical ingredient is selected from the group consisting of an essence oil isolated from a citrus fruit, a peel oil isolated from a citrus fruit, a peel isolated from a citrus fruit, decharacterized citrus fruit, and combinations thereof.

13. The functional food according to claim 10, comprising from about 10 mg to about 80 mg of the tocotrienol and from about 150 mg to about 750 mg of the flavonoid per serving.

14. The functional food according to claim 10, comprising about 30 mg of the tocotrienol and about 270 mg of the flavonoid per serving.

15. The functional food according to claim 10, comprising about 60 mg of the tocotrienol and about 560 mg of the flavonoid per serving.

16. The functional food according to claim 10, in the form of a chewable or edible bar, a confectionary product, a cookie, a juice drink, a puree, a baked or simulated baked good, a biscuit, a lozenge or chewing gum.

17. The functional food according to claim 10, in the form of a brownie or a chocolate bar.

18. The functional food according to claim 10, in the form of butter, margarine, bread, cake, a milk shake, ice cream, yogurt or other fermented milk product.

19. The functional food according to claim 10, in the form of a powder or a cereal.

20. A method of treating a human subject at risk of or suffering from a neoplastic disease comprising administering an effective amount of a pharmaceutical formulation according to claim 1.

21. A method of treating a human subject at risk of or suffering from inflammation comprising administering an effective amount of a pharmaceutical formulation according to claim 2.

22. A method of treating a human subject at risk of or suffering from a neoplastic disease comprising administering an effective amount of a functional food according to claim 10 any.

23. A method of treating a human subject at risk of or suffering from inflammation comprising administering an effective amount of a functional food according to claim 11.
